(19) United States
(12) Patent Application Publication
(10) Pub. No.: US 2008/0082060 A1
(43) Pub. Date: Apr. 3, 2008

(54) Cord blood correcting device and method of correcting cord blood

(76) Inventors: Hajime Ogata, Osaka (JP); Hideaki Murahashi, Osaka (JP); Kazuyuki Takeda, Osaka (JP); Akio Shirasu, Osaka (JP); Yoshihiro Yoshikawa, Osaka (JP)

Correspondence Address: KUBOVCIK & KUBOVCIK, SUITE 710, 900 17TH STREET NW, WASHINGTON, DC, 20006, US

(21) Appl. No.: 11/905,093

(22) Filed: Sep. 27, 2007

(30) Foreign Application Priority Data
Sep. 28, 2006 (JP) 2006-265684

(51) Int. Cl. A61M 1/00; A61B 19/00

(52) U.S. Cl. 604/317; 604/408

(57) ABSTRACT

The present invention relates to a cord blood correcting device characterized by including: a branch indwelling needle having an outer cylinder needle provided on a distal end side, an inner needle drawing tube portion provided on a proximal end side, an inner needle provided inside the inner needle drawing tube portion, and a liquid outflow tube portion which branches off from the inner needle drawing tube portion; a cord tissue holder having a base portion and a holding portion for holding the cord tissue; and connecting means for interconnecting the branch indwelling needle and the cord tissue holder, and in that the connecting means has a structure for changing an angle between a puncture direction from the proximal end side to the distal end side of the branch indwelling needle and a holding direction from the base portion to the holding portion of the cord tissue holder.

### BACKGROUND OF THE INVENTION

1. Field of the Invention

The present invention relates to a cord blood correcting device, and more particularly, to a cord blood correcting device including a branch indwelling needle having an outer cylinder needle provided on the distal end side, an inner needle drawing tube portion provided on the proximal end side, an inner needle provided inside the inner needle drawing tube portion, and a liquid outflow tube portion which branches off from the inner needle drawing tube portion, a cord tissue holder having a base portion and a holding portion for holding a cord tissue, and connecting means for interconnecting the branch indwelling needle and the cord tissue holder, in which the connecting means has a structure for changing the angle between a puncture direction from the proximal end side to the distal end side of the branch indwelling needle and a holding direction from the base portion to the holding portion of the cord tissue holder. The present invention also relates to a cord blood correcting method using the device.

2. Description of the Related Art

In recent years, approximately 100,000 premature babies having a birth weight of 2,500 g or less have been born each year in Japan. Almost all of these premature babies, especially very low birth weight infants (VLBWI) having a birth weight of 1,500 g or less are affected with anemia due to frequent blood correcting or drastic weight increase in a neonatal intensive cure unit (NICU). This disease is recognized as anemia of prematurity and has been treated by blood transfusion. Until the beginning of 1990, about 40% of VLBWI's in Japan and about 60% to 80% of VLBWI's in US and Europe had received blood transfusion frequently. The main symptoms of the anemia of prematurity include apneic attack, rapid respiration, rapid beat, reduction in feed force, failure in gaining weight, and hypoimmunity.

For the treatment of the anemia of prematurity, use of a recombination human erythropoietin preparation which selectively shows strong enhancing action for erythrocyte hematogenesis has been authorized, and thus the number of blood transfusions has been decreasing. However, this cannot eliminate blood transfusion itself completely.

In Japan, blood transfusion guidelines for early anemia of prematurity have been formulated by the Infant Transfusion Therapy Research Association which was founded in 1992, and guidelines for the use of blood preparations for children have been notified by the Pharmaceutical and Medical Safety Bureau in 1999.

However, the allogeneic transfusion of the blood of an adult into a premature baby is one type of organ transplant, and side-effects caused by blood transfusion such as viral infections by the window period including serum hepatitis and AIDS and graft-versus-host diseases are unavoidable for premature babies who are immunologically premature and have low power of resistance. In order to avoid side effects caused by allogeneic transfusion, storage type autologous transfusion is generally employed for adults.

Meanwhile, autologous cord blood transfusion treatment, which does not use adult human blood but cord blood, is proposed for premature babies. Since the oxygen dissociation curve of cord blood moves in a left-hand direction more than that of adult human blood, its oxygen transport ability under a hypoxic condition is high, which is advantageous not only for respiration in peripheral tissues. This is advantageous for the development and growth of organs including the central nervous system in the actual environment of an unborn baby having a low oxygen partial pressure of around 30 mmHg, but also for avoiding hypoxic-ischemic organ derangement caused by various stresses.

The inventors of the present invention have verified the efficacy of the above treatment by carrying out the autologous cord blood transfusion treatment many times. Specifically, the number of apneic attacks and developmental delay are reduced and a load on the circulatory system reduces, whereby the duration of hospitalization is shortened. Therefore, the autologous cord blood transfusion treatment is expected to spread worldwide as universal treatment with which allogeneic transfusion can be avoided in the future.

Meanwhile, because the cord blood contains a large number of stem cells, it is used for the graft treatment of leukemia or the like. This treatment called “cord blood graft” has an advantage in that there is no attack on a donor like marrow graft, and is already spreading worldwide as universal treatment.

Various studies have been made on cord blood correcting and preservation technologies in order to use them for these treatments. The general methods of correcting cord blood are given below.
(A) A cord blood vessel is punctured with a blood collecting needle to collect cord blood by the suction force of a syringe (JP 07-184991 A1).
(B) A placenta holder disclosed in Patent JP 10-108869 A1, JP 10-155809 A1, JP 11-009576 A and JP 11-033016 A1 is used to hang the cord, and the cord blood vessel is punctured with a blood collecting needle to collect cord blood by using its drop.

Since the blood vessel may get crushed and be blocked when the cord blood is sucked rapidly in the method (A), the method (B) in which the cord blood is collected by using its drop is preferred.

However, since blood correcting is carried out while the cord is hung in the conventional method as described above, the blood vessel may be damaged by a change in the relative positions of the blood correcting device and the vein of the cord when the blood correcting device is inserted into the vein of the cord. Therefore, the blood may leak and/or spatter.

Although blood correcting work is carried out right after a baby is delivered, because top priority is given to taking care of a newborn baby and a mother, the correcting of cord blood needs to be carried out as simply and surely as possible. However, in the conventional method, an operator must hold a cord blood correcting device and the cord by hand to fix the direction of inserting the needle. Because the operator is thus kept under restraint while correcting cord blood, care for a mother and a baby is greatly restricted. In the conventional method, the inner cylinder of the collecting needle may be stuffed with cord tissues during the correcting work of the cord blood, or the mouth portion at the end of the collecting needle may be blocked by the wall of the blood vessel. In this case, although the blood correcting efficiency is improved by changing the direction of inserting the needle, the blood vessel may be damaged when the direction is changed.

### BRIEF SUMMARY OF THE INVENTION

Thus, the development of a cord blood correcting device which does not damage the blood vessel of the cord when cord blood is corrected, enables stable blood correcting, and enables an operator to concentrate on taking care of a premature baby and a mother without anxiety is desired.

The present invention provides the following.
[1] a cord blood correcting device for correcting cord blood from a cord tissue separated from a human being, characterized by including:

a branch indwelling needle having an outer cylinder needle provided on a distal end side, an inner needle drawing tube portion provided on a proximal end side, an inner needle provided inside the inner needle drawing tube portion, and a liquid outflow tube portion which branches off from the inner needle drawing tube portion;

a cord tissue holder having a base portion and a holding portion for holding the cord tissue; and

connecting means for interconnecting the branch indwelling needle and the cord tissue holder,

and in that the connecting means has a structure for changing an angle between a puncture direction from the proximal end side to the distal end side of the branch indwelling needle and a holding direction from the base portion to the holding portion of the cord tissue holder.
[2] The cord blood correcting device according to Item [1], characterized in that:

the base portion of the cord tissue holder is provided with a connection portion to be directly connected to the branch indwelling needle; and

the branch indwelling needle is provided with angle changing means.
[3] The cord blood correcting device according to Item [2], characterized in that the angle changing means is a cylindrical body or a groove which has an angle formed between the branch indwelling needle in the puncture direction and is engaged with the connection portion of the cord tissue holder.
[4] The cord blood correcting device according to Item [3], in which the angle is 45 to 135°.
[5] The cord blood correcting device according to Item [1], characterized by further including a connection aid for indirectly interconnecting the branch indwelling needle and the cord tissue holder,

and in that the angle changing means is provided to the connection aid.
[6] The cord blood correcting device according to Item [1], characterized by further including a blood bag for storing collected cord blood.
[7] A method of correcting cord blood from a cord tissue separated from a human being by using a cord blood correcting device, the device including:

a branch indwelling needle having an outer cylinder needle provided on a distal end side, an inner needle drawing tube portion provided on a proximal end side, an inner needle provided inside the inner needle drawing tube portion, and a liquid outflow tube portion which branches off from the inner needle drawing tube portion;

a cord tissue holder having a base portion and a holding portion for holding the cord tissue; and

connecting means for interconnecting the branch indwelling needle and the cord tissue holder,

the connecting means having a structure for changing an angle between a puncture direction from the proximal end side to the distal end side of the branch indwelling needle and a holding direction from the base portion to the holding portion of the cord tissue holder,

the method including changing, by the connecting means, the angle formed between the puncture direction and the holding direction when the cord tissue is punctured with the branch indwelling needle.

Since the cord blood correcting device of the present invention enables for the stable correcting of cord blood, an operator can concentrate on taking care of a premature baby and a mother. Specifically, since a branch indwelling needle having a plastic outer cylinder needle for correcting cord blood is fixed to an insertion position by using a cord tissue holder so as to correct the cord blood, the risk of damaging the blood vessel during the operation of correcting the blood is low and precious cord blood does not spatter.

### BRIEF DESCRIPTION OF THE DRAWINGS

FIG. 1 is a diagram showing an embodiment of a device of the present invention.

FIG. 2 is a diagram showing a branch indwelling needle in the device of FIG. 1.

FIG. 3 is a diagram showing a cord tissue holder in the device of FIG. 1.

FIG. 4 is a diagram showing a puncture of a cord using the device of FIG. 1.

FIG. 5 is a diagram showing that the angle can be changed in the device of FIG. 1.

FIG. 6 is a diagram showing a modification of the branch indwelling needle of FIG. 2.

FIG. 7 is a diagram showing another embodiment different from the device of FIG. 1.

FIG. 8 is a diagram showing a branch indwelling needle in the device of FIG. 7.

FIG. 9 is a diagram showing a cord tissue holder in the device of FIG. 7.

FIG. 10 is a diagram showing that the angle can be changed in the device of FIG. 7.

FIG. 11 is a diagram showing still another embodiment different from the devices of FIGS. 1 and 7.

FIG. 12 is a diagram showing an embodiment further including a blood bag in the device of FIG. 1.

1 branch indwelling needle

2 cord tissue holder

3 connecting means

4 blood bag

5 tube

11 distal end side

12 proximal end side

13 inner needle

14 liquid outflow tube portion

21 holding portion

22 base portion

23 connection aid

61 communication piece

62 liquid flow control means

111 outer cylinder needle

121 inner needle drawing tube portion

311 cylindrical body

312 grasping portion

313 groove

321 spherical body

322 grasping body

331 stepwise angle changing means

X cord

### DETAILED DESCRIPTION OF THE INVENTION

The present invention will be described inbelow with reference to the accompanying drawings.

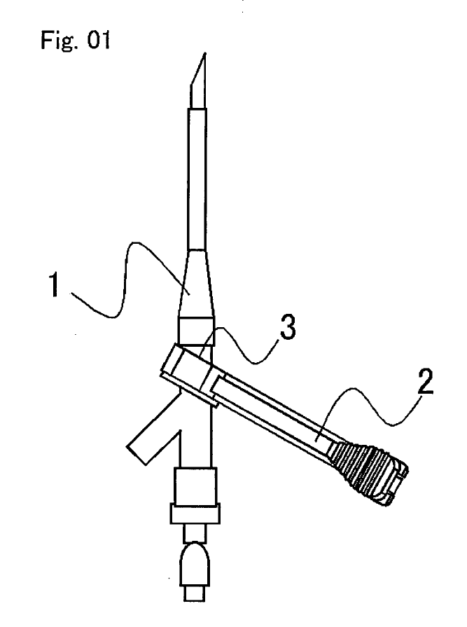

FIG. 1 is a schematic diagram of the cord blood correcting device of the present invention. The cord blood correcting device of the present invention includes a branch indwelling needle 1, a cord tissue holder 2, and angle changing means 3. The cord tissue holder 2 is connected to the branch indwelling needle 1.

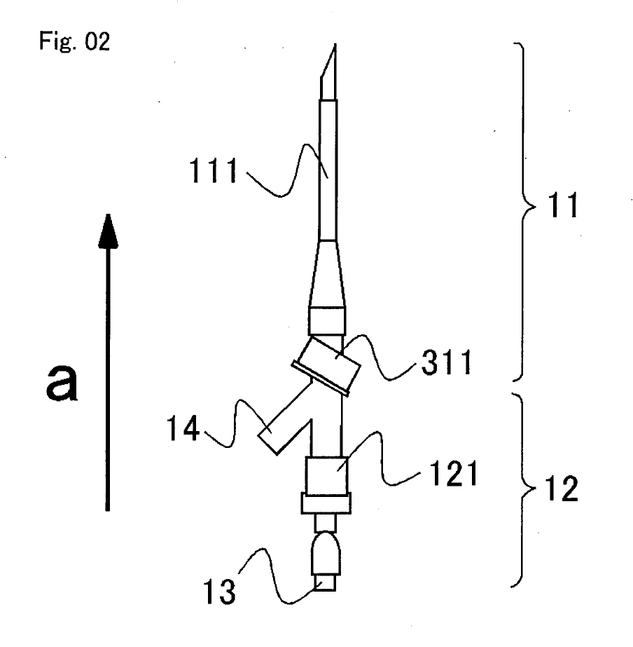

FIG. 2 shows the details of the branch indwelling needle 1 shown in FIG. 1. The branch indwelling needle 1 of the present invention is a device constituted such that a tissue is punctured with a metal inner needle and then a plastic outer cylinder needle is left in the blood vessel of the cord by pulling out the metal inner needle. Specifically, the branch indwelling needle 1 includes an outer cylinder needle 111 provided on the distal end side 11, an inner needle drawing tube portion 121 provided on the proximal end side 12, an inner needle 13 provided inside the inner needle drawing tube portion 121, and a liquid outflow tube portion 14 which branches off from the inner needle drawing tube portion 121. In the present invention, the direction (shown by an arrow “a” in FIG. 2) from the proximal end side 12 to the distal end side 11 of the branch indwelling needle 1 is designated as a “puncture direction”,

The basic structure of the branch indwelling needle 1 is the same as that of a commercially available branch indwelling needle. The outer cylinder needle 111, the inner needle drawing tube portion 121, and the liquid outflow tube portion 14 are made of plastic, and the inner needle 13 is made of metal such as stainless steel, aluminum, aluminum alloy, titanium, or titanium alloy. The above plastic is preferably a soft resin having excellent biocompatibility, antibiotic properties, and flexibility, such as polyurethane or ethylene-polytetrafluoroethylene copolymer, but the present invention is not limited to these. Since the outer cylinder needle 111 is made of plastic, the blood vessel is not damaged by the blood correcting operation and precious cord blood does not spatter. A metal needle is commonly used in the technical field in which cord blood is corrected from a cord tissue, and a branch indwelling needle is not used in the general medical field. The inventors of the present invention have found a problem in that a metal needle penetrates or rips the blood vessel by an impulse from a blood flow or during blood correcting operation, and have solved this problem.

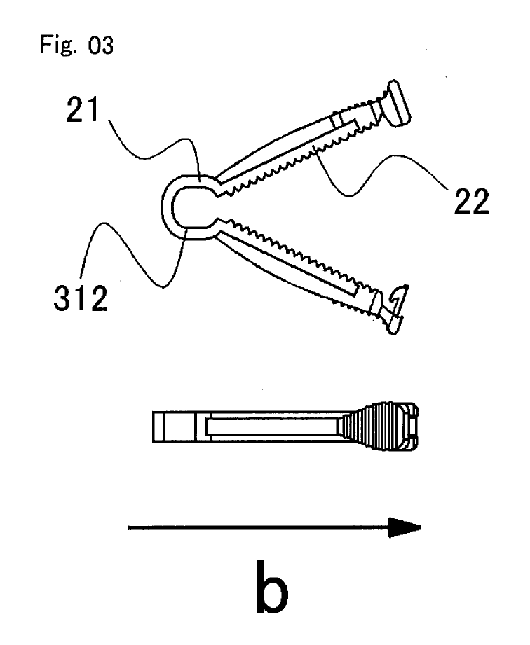

FIG. 3 shows an example of the details of the cord tissue holder 2 shown in FIG. 1. The cord tissue holder 2 of the present invention includes a base portion 21 which is directly or indirectly connected to the branch indwelling needle 1 and a holding portion 22 for holding a cord tissue. The cord tissue holder 2 is a device for holding the branch indwelling needle 1 in the cord tissue so that the branch indwelling needle 1 does not slip out from the cord tissue while the cord blood is corrected. The structure of the holding portion 22 of the cord tissue holder 2 is not particularly limited as long as it has holding force with which the branch indwelling needle 1 does not slip out from the cord tissue and the blood vessel of the cord is not crushed. Although the cord tissue may be sandwiched, grasped, bit or held, it is preferably sandwiched as shown in FIG. 3 from the viewpoints of easiness in manufacture and low costs. In the present invention, the direction (shown by an arrow “b” in FIG. 3) from the base portion 21 to the holding portion 22 which is parallel to a straight line having the shortest distance between the base portion 21 and the holding portion 22 of the cord tissue holder 2 is designated as a “holding direction”.

The branch indwelling needle 1 and the cord tissue holder 2 are interconnected by connecting means 3. For instance, connection is effected by engaging the cylindrical body 311 of the branch indwelling needle 1 shown in FIG. 2 with a grasping portion 312 provided in the base portion 21 of the cord tissue holder 2 shown in FIG. 3. The structure of the connecting means 3 is such that connection is effected directly by engagement, fitting, welding, sandwiching, holding, biting, or grasping between the member of the branch indwelling needle 1 and the member of the cord tissue holder 2, or indirectly by means of a member for interconnecting the branch indwelling needle 1 and the cord tissue holder 2. The structure of the connecting means is not particularly limited since it can be suitably designed by the person skilled in the art.

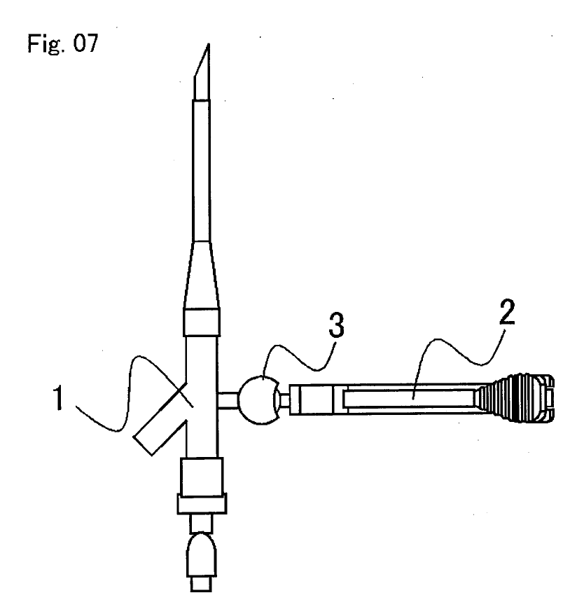

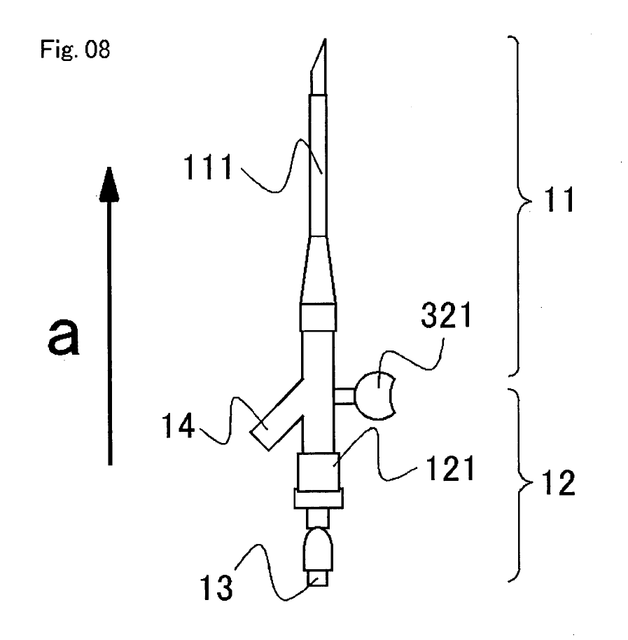

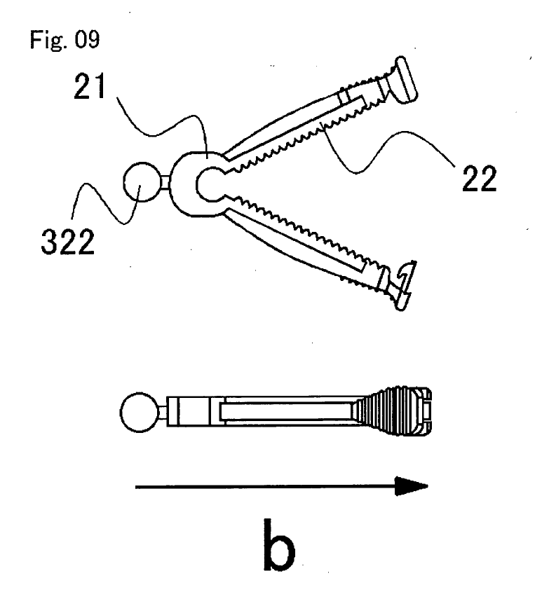

For example, FIGS. 7 to 9 show a modifications which are different from the connecting means 3 shown in FIGS. 1 to 3 in structure. The branch indwelling needle 1 of FIG. 8 has a spherical body 321 on the side wall of the inner needle drawing tube portion 121. Meanwhile, the cord tissue holder 2 of FIG. 9 includes a grasping body 322 which has a structure for grasping the spherical body 321, and the branch indwelling needle 1 and the cord tissue holder 2 are interconnected by allowing the grasping body 322 to be held in the spherical body 321 of the branch indwelling needle 1.

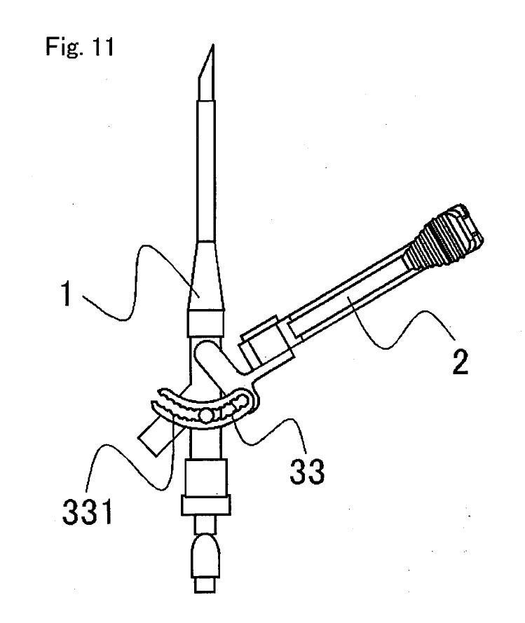

Further, as shown in FIG. 11, there is a modification which further includes a connection aid 33 for indirectly interconnecting the branch indwelling needle 1 and the cord tissue holder 2.

The connecting means 3 may be provided on the proximal end side 12 or the liquid outflow tube portion 14 of the branch indwelling needle 1 and/or any position on the cord tissue holder 2. It is preferably provided on the proximal end side 12 of the branch indwelling needle 1 as it is easy to puncture the cord tissue, or on the base portion 22 of the cord tissue holder 2 as it is easy to hold the cord tissue. The cord tissue holder 2 may be detachable from the branch indwelling needle 1.

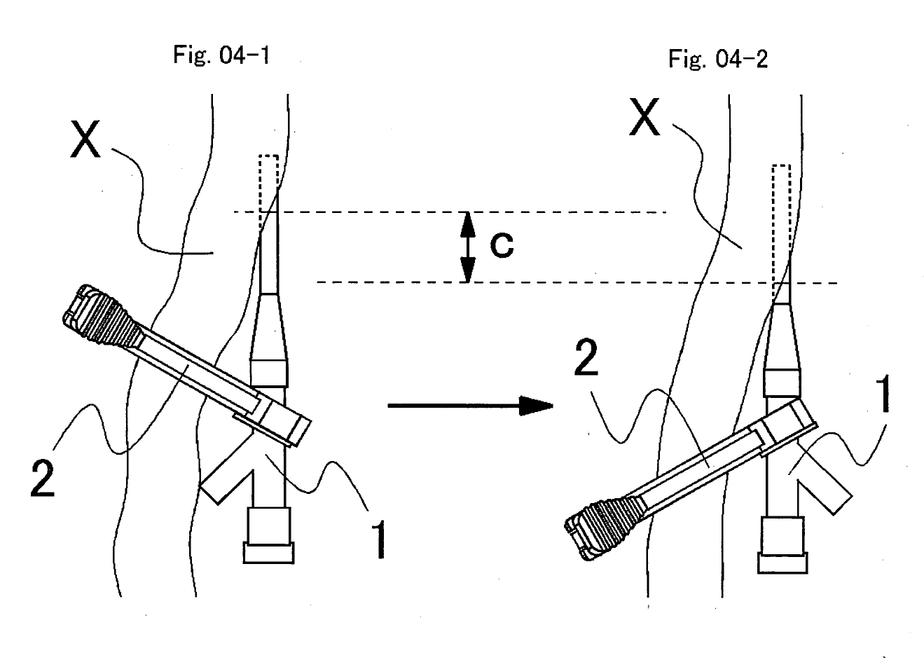

The connecting means 3 has a structure for changing the angle between the branch indwelling needle 1 and the cord tissue holder 2. Specifically, it has a structure for changing the angle between the puncture direction and the holding direction as well as the direction of puncturing the cord tissue with the branch indwelling needle 1. With this structure, after the insertion and indwelling of the outer cylinder needle 111 in the blood vessel of the cord, the outer cylinder needle 111 is fixed to the cord by using the cord tissue holder 2 on the puncture direction “a” side as shown in FIG. 4-1. After that, the branch indwelling needle 1 is turned 180° by the connecting means 3 to change its direction as shown in FIG. 4-2. Accordingly, the outer cylinder needle 111 is inserted deep into the blood vessel of the cord by a distance C, with the result that the leakage of the cord blood from the base portion of the outer cylinder needle 111 can be prevented. When the outer cylinder needle 111 becomes thicker from its end toward its base, it has a larger effect of preventing the leakage of the cord blood. If the side wall of the blood vessel of the cord tissue blocks the mouth portion of the outer cylinder needle 111 of the branch indwelling needle 1, the cord blood can be corrected more efficiently by changing the puncture direction into the cord tissue by means of the connecting means 3.

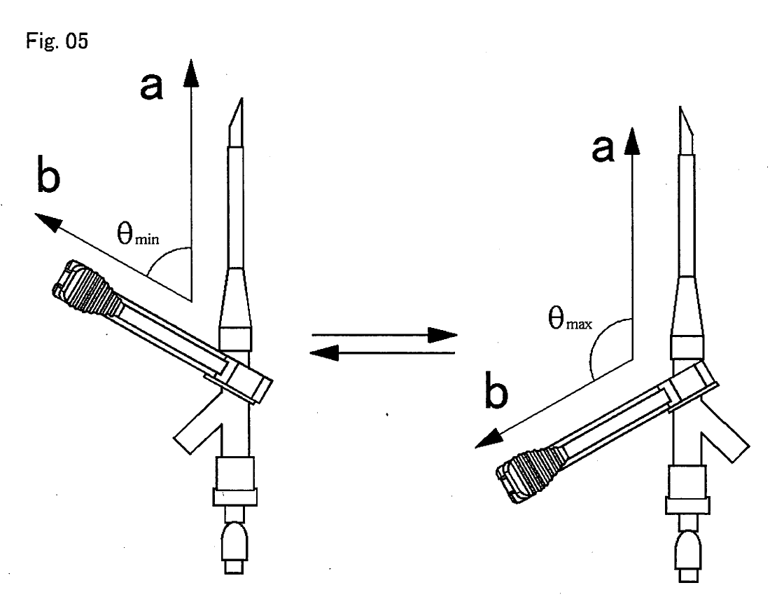

For example, the cylindrical body 311 may be provided at an angle from the puncture direction like the branch indwelling needle 1 shown in FIG. 2. The cylindrical body 311 can change the angle between the puncture direction “a” and the holding direction “b” by turning the branch indwelling needle 1 while it engages with the grasping portion 312 of the cord tissue holder 2 shown in FIG. 3 (FIG. 5). The angle between the puncture direction “a” and the holding direction “b” is fixed by friction resistance between the cylindrical body 311 and the grasping portion 312 as long as artificial force is not applied. This makes possible the stable correcting of the cord blood. Although the angle of the cylindrical body 311 is not particularly limited, it is about 5 to 45°, preferably about 10 to 30° from the viewpoint of the easiness in manufacture of the cylindrical body 311. That is, when the angle of the cylindrical body is 50, the angle between the puncture direction and the holding direction becomes 80 to 95°. When the angle of the cylindrical body is 300, the angle between the puncture direction and the holding direction becomes 60 to 120°. When the angle of the cylindrical body is 45°, the angle between the puncture direction and the holding direction becomes 45 to 135°.

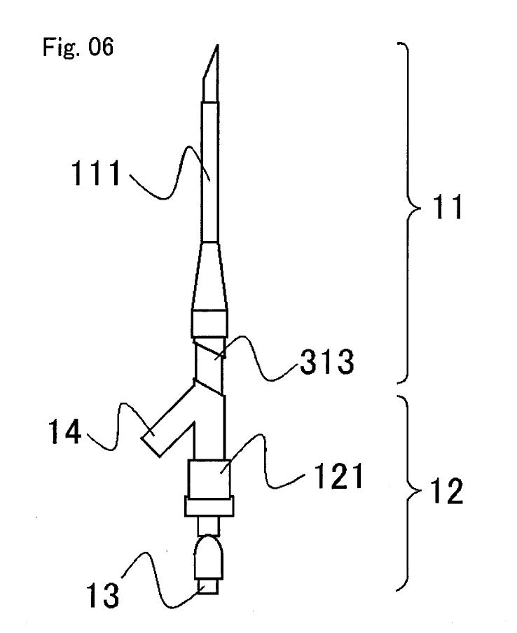

Changing the angle between the puncture direction and the holding direction by means of a groove 313 shown in FIG. 6 and not by means of the cylindrical body 311 shown in FIG. 2 could have been easily realized by the person skilled in the art.

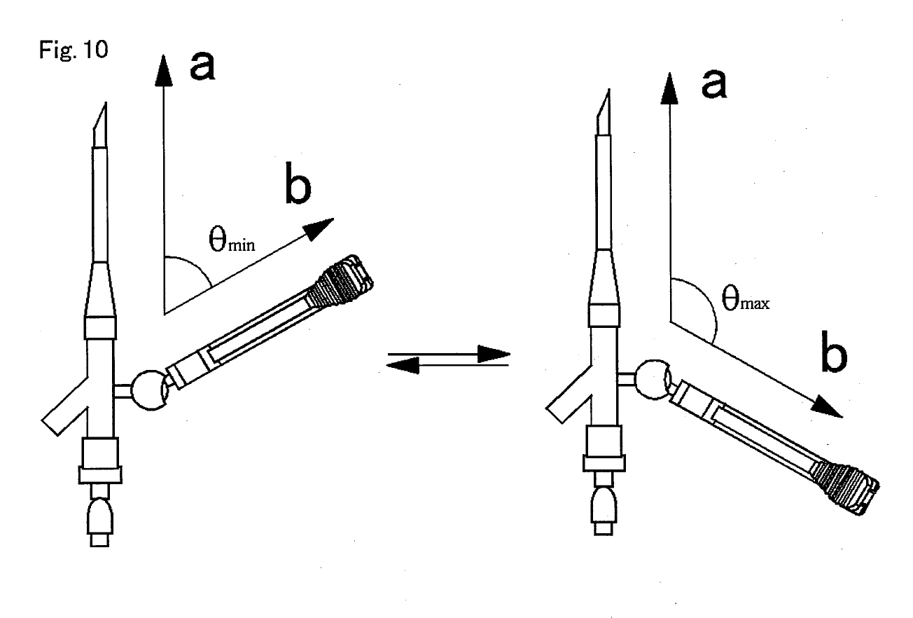

Further, in the structure of the connecting means 3 shown in FIGS. 7 to 9, the angle between the puncture direction and the holding direction can be changed by engaging the spherical body 321 of the branch indwelling needle 1 shown in FIG. 8 with the grasping body 322 of the cord tissue holder 2 shown in FIG. 9 (FIG. 10). The angle between the puncture direction and the holding direction is fixed by friction resistance between the spherical body 321 and the grasping body 322 as long as artificial force is not applied. This makes possible the stable correcting of the cord blood.

In addition, in the structure of the connecting means 3 shown in FIG. 11, the angle between the puncture direction and the holding direction can be changed by a stepwise angle changing structure 331 provided in the connection aid 33. The angle between the puncture direction and the holding direction is fixed by the stepwise angle changing means 331 as long as artificial force is not applied. This makes possible the stable correcting of the cord blood.

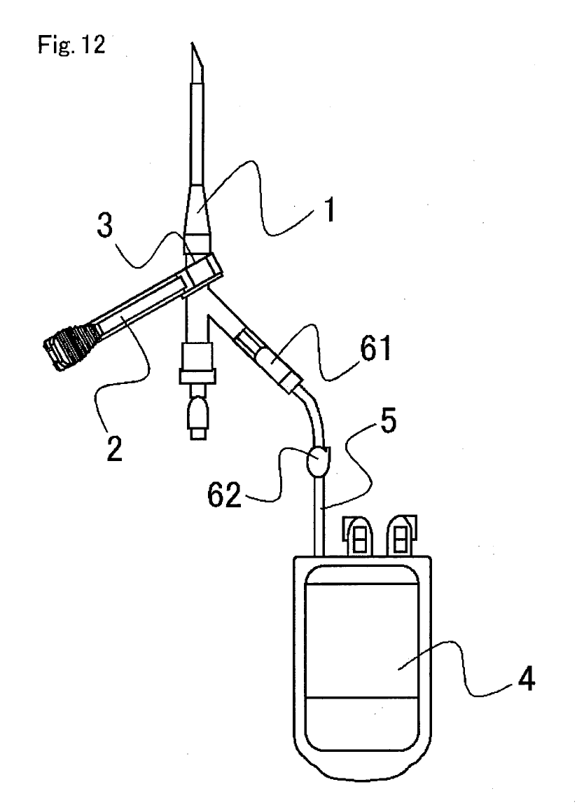

Preferably, the cord blood correcting device of the present invention further includes a blood bag 4 as shown in FIG. 12. The blood bag 4 is connected to the liquid outflow tube portion 14 of the branch indwelling needle 1 in advance. Therefore, after the cord tissue is punctured, cord blood can be introduced into the blood bag aseptically. The term “connection” in the present invention means internal communication, generally, connection by means of a tube 5. The tube 5 may be made of the same material as that of a general-purpose medical tube, for example, a polyvinyl chloride tube. The material of the blood bag may be the same as that of a known blood bag, such as polyethylene or polyvinyl chloride. The capacity of the above blood bag 4 is about 100 to 400 ml, preferably about 150 to 250 ml when the following is taken into consideration: the amount of the corrected cord blood during the delivery of a premature baby is about 40 to 150 ml in the case of vaginal delivery; and a drug solution which will be described later is pre-stored in the blood bag 4 and mixed with the corrected cord blood.

Further, the above blood bag may contain a drug solution suitable for the preservation of the blood. Examples of the drug solution include whole blood preparation (Japan standard commodity classification No: 876341), erythrocyte preservative solution (also called “biological product standard blood preservative solution A”, Japan standard commodity classification No: 873339), and blood component preparation (Japan standard commodity classification No: 876342) which can be used discriminately according to its purpose. The amount of the drug solution is about 10 to 60 ml, preferably about 20 to 30 ml from the viewpoint of its influence upon the human body at the time of blood transfusion. The tube 5 may be provided with liquid flow control means 6 such as a communication piece and/or a clamp. Since these means related to the connection of the blood bag can be suitably designed by the person skilled in the art, they are not particularly limited.

### EXAMPLE

The method of correcting cord blood with the cord blood correcting device shown in FIG. 12 of the present invention will be described hereinbelow. However, the present invention is not limited to this.

### EXAMPLE 1

### Correcting of Cord Blood

In this example, the approval of the ethical committee of an insurance medical care facility and substantial informed consent from the guardian of a patient are necessary. The term “informed consent” in the present invention means that a sufficient explanation of autologous cord blood transfusion treatment has been given to the guardian of a patient and a consent has been obtained through self-decision of the guardian with satisfaction. Thus an ethical problem has been solved.

(1) Two positions at the end on the corded baby side are clamped before the expulsion of a placenta after the delivery of a baby, and the section between them is cut to take out the baby. In the case of C-section, after the separation of the baby, the placenta is removed without damaging the blood capillary and is transferred to a kidney dish.
(2) To suppress the risk of the bacterial infection of cord blood, the cord is cleaned with gauze impregnated with external antiseptic (Japan standard commodity classification No: 872612) from the end on the baby side toward the placenta side and then with clean dry gauze similarly.
(3) The separated placenta and the separated cord are set so that the cord blood can be corrected by its drop. For example, a placenta holder as disclosed in JP 10-108869 A, JP 10-155809 A, JP 11-009576 A, and JP 11-033016 A is preferably used to hang the cord. However, the present invention is not limited to this.
(4) The vein of the cord is punctured with the branch indwelling needle 1.
(5) The inner needle 13 is pulled out.
(6) The branch indwelling needle 1 is turned (by controlling the connecting means 3) to locate the cord tissue holder 2 on the distal end side.
(7) The cord is fixed by the cord tissue holder 2.
(8) The branch indwelling needle 1 is turned (by controlling the connecting means 3) to locate the cord tissue holder 2 on the proximal end side and the outer needle 111 is inserted deep into the blood vessel of the cord (blood leakage becomes unlikely to happen).
(9) The communication piece 61 is bent to start blood correcting.
(10) The branch indwelling needle 1 is turned (by controlling the connecting means 3) to adjust the angle and find a suitable puncture angle.
(11) Liquid flow control means 62 is clamped to terminate blood correcting. When a placenta holding apparatus disclosed in JP 11-009576 A and JP 11-033016 A is used at this point, the blood remaining in the placenta can be visually observed with ease, which is convenient. When a dusky-red portion of the placenta is pressed, the cord blood can be corrected without waste.

### Test Example 1

### Comparison of Amount of Corrected Blood

Cord blood was corrected by using the cord blood correcting device of the present invention. Specifically,
(1) two positions at the end on the corded baby side were clamped before the expulsion of a placenta after the delivery of a baby, and the section between them was cut to take out the baby. In the case of C-section, after the separation of the baby, the placenta was removed without damaging the blood capillary and was transferred to a kidney dish.
(2) To suppress the risk of the bacterial infection of cord blood, the cord was cleaned with gauze impregnated with external antiseptic (Japan standard commodity classification No: 872612) from the end on the baby side toward the placenta side and then with clean dry gauze similarly.
(3) The separated placenta and the separated cord were set so that the cord blood could be corrected by a head drop.
(4) The vein of the cord was punctured with the branch indwelling needle 1.
(5) The inner needle 13 was pulled out. (6) The branch indwelling needle 1 was turned (by controlling the connecting means 3) to locate the cord tissue holder 2 on the distal end side.
(7) The cord was fixed by the cord tissue holder 2.
(8) The branch indwelling needle 1 was turned (by controlling the connecting means 3) to locate the cord tissue holder 2 on the proximal end side and the outer needle 111 was inserted deep into the blood vessel of the cord (blood leakage becomes unlikely to happen).
(9) The communication piece 61 was bent to start blood correcting.
(10) The branch indwelling needle 1 was turned (by controlling the connecting means 3) to find a suitable puncture angle.
(11) Liquid flow control means 62 was clamped to terminate blood correcting.

It was confirmed that the above blood correcting work could be carried out very stably. The amount of the corrected cord blood was 95.1 ml. This amount was larger than the amount of the conventionally corrected cord blood (70.4 ml, refer to the web site “Jump into medical science of the 21st century, lecture of clinical medicine, lecture of pediatrics”, URL: http://www2.kmu.ac.jp/ann70/tenkai/clin/text8.html). The utility of the present invention has been verified also from this result.

What is claimed is:

1. A cord blood correcting device for correcting cord blood from a cord tissue separated from a human being, characterized by comprising:
a branch indwelling needle having an outer cylinder needle provided on a distal end side, an inner needle drawing tube portion provided on a proximal end side, an inner needle provided inside the inner needle drawing tube portion, and a liquid outflow tube portion which branches off from the inner needle drawing tube portion;
a cord tissue holder having a base portion and a holding portion for holding the cord tissue; and
connecting means for interconnecting the branch indwelling needle and the cord tissue holder,
and in that the connecting means has a structure for changing an angle between a puncture direction from the proximal end side to the distal end side of the branch indwelling needle and a holding direction from the base portion to the holding portion of the cord tissue holder.

2. The cord blood correcting device according to claim 1, characterized in that:
the base portion of the cord tissue holder is provided with a connection portion to be directly connected to the branch indwelling needle; and
the branch indwelling needle is provided with angle changing means.

3. The cord blood correcting device according to claim 2, characterized in that the angle changing means comprises a cylindrical body or a groove which has an angle formed between the branch indwelling needle in the puncture direction and is engaged with the connection portion of the cord tissue holder.

4. The cord blood correcting device according to claim 3, characterized in that the formed angle is 45 to 135°.

5. The cord blood correcting device according to claim 1, characterized by further comprising a connection aid for indirectly interconnecting the branch indwelling needle and the cord tissue holder,
and in that the angle changing means is provided to the connection aid.

6. The cord blood correcting device according to claim 1, characterized by further comprising a blood bag for storing collected cord blood.

7. A method of correcting cord blood from a cord tissue separated from a human being by using a cord blood correcting device, the device including:
a branch indwelling needle having an outer cylinder needle provided on a distal end side, an inner needle drawing tube portion provided on a proximal end side, an inner needle provided inside the inner needle drawing tube portion, and a liquid outflow tube portion which branches off from the inner needle drawing tube portion;
a cord tissue holder having a base portion and a holding portion for holding the cord tissue; and
connecting means for interconnecting the branch indwelling needle and the cord tissue holder,
the connecting means having a structure for changing an angle between a puncture direction from the proximal end side to the distal end side of the branch indwelling needle and a holding direction from the base portion to the holding portion of the cord tissue holder,
the method comprising changing, by the connecting means, the angle formed between the puncture direction and the holding direction when the cord tissue is punctured with the branch indwelling needle.
